(19) United States
(12) Patent Application Publication
(10) Pub. No.: US 2023/0226241 A1
(43) Pub. Date: Jul. 20, 2023

(54) SANITISATION SYSTEM AND METHOD

(71) Applicant: Pandemic Advisory & Audit Pty Ltd, Camp Hill, QLD (AU)

(72) Inventors: John Focas, Brisbane,QLD (AU); Martin Morrow-Woods, Brisbane,QLD (AU)

(21) Appl. No.: 18/001,195

(22) PCT Filed: Jun. 8, 2021
(86) PCT No.: PCT/AU2021/050570

§ 371 Date: Dec. 8, 2022

(30) Foreign Application Priority Data
Jun. 8, 2020 (AU) 2020901877

(51) Int. Cl. A61L 9/03

(52) U.S. Cl. CPC A61L 9/032; A61L 2209/211; A61L 2209/135; A61L 2209/111

(57) ABSTRACT

A sanitisation system comprising a chemical vaporising apparatus for storing and vaporising a sanitising chemical com- position. The chemical vaporising assembly comprising a housing configured to receive and retaining one or more sanitising chemical composition containing cartridges, a vaporiser located within the housing, the vaporiser configured to receive and convert a sanitising chemical composition in the chemical containing cartridges into a vapor, and an air purifier assembly located within the housing, the air purifier assembly configured to purify the air by removing vaporised sanitising chemical composition from the air and maintain a low humidity environment.

### TECHNICAL FIELD

The invention described herein relates to a sanitisation system and method. In particular, the invention relates to a chemical dispersion system for sanitising common public areas to reduce or prevent the transmission of bacteria and disease through contact with germ carrying surfaces.

### BACKGROUND

Any reference to methods, apparatus or documents of the prior art are not to be taken as constituting any evidence or admission that they formed, or form, part of the common general knowledge.

Many sanitisation and decontamination practices rely on humans to physically apply decontaminating and sanitising chemical compositions to objects and surfaces. These existing practices are prone to error (through honest mistake and carelessness) and the efficacy of such practices cannot be properly measured or quantified. In times of pandemic or in the face of highly infection diseases, this can be highly problematic and may lead to increased levels of infection.

### OBJECT

It is an aim of this invention to provide a sanitisation system and/or method which overcomes or ameliorates one or more of the disadvantages or problems described above, or which at least provides a useful commercial alternative.

Other preferred objects of the present invention will become apparent from the following description.

### SUMMARY OF THE INVENTION

In one aspect, although it need not be the only or indeed the broadest aspect, there is provided a sanitisation system, the sanitisation system comprising:
a chemical vaporising apparatus for storing and vaporising a sanitising chemical composition, the chemical vaporising assembly comprising:
   a housing configured to receive and retaining one or more cartridges containing a sanitising chemical composition;
   an air purifier assembly located within the housing, the air purifier assembly configured to purify the air by removing vaporised sanitising chemical composition and maintain a low humidity environment; and
   a vaporiser located within the housing, the vaporiser configured to receive and convert a sanitising chemical composition in the chemical containing cartridges into a vapor; an outlet assembly comprising:
   a duct connected to the chemical vaporising assembly for transporting the vapor therein;
   an elongate dispersion member having a first end connected to the duct, the elongate dispersion member having a plurality of apertures formed therein for dispersing vapor therethrough; and
   a fan located at a second end of the elongate dispersion member distal to the first end of the elongate dispersion member for drawing air into the dispersion member; and
a system controller programmed to activate the fan to draw air into the elongate dispersion member for mixing with the vapor and dispersal through the plurality of apertures formed in the elongate dispersion member.

In another aspect, there is provided a method for sanitisation, the method comprising the steps of:
generating a vapor from a sanitising chemical composition by a vaporiser in a housing;
transporting the vapor to a dispersion member having a plurality of apertures formed therein; and
activating a fan to draw air into the dispersion member to mix the air and the vapor and disperse the mixture into a surrounding space through the apertures.

Preferably, the sanitising chemical composition comprises a sanitising chemical. Preferably, the sanitising chemical comprises hydrogen peroxide. Preferably, the sanitising chemical composition comprises up to 35% mL/mL hydrogen peroxide. More preferably, the sanitising chemical composition comprises between 0.1% mL/mL and 35% mL/mL hydrogen peroxide. Preferably, the sanitising chemical composition comprises no more than 10% mL/mL hydrogen peroxide. Alternatively, the sanitising chemical composition comprises ionic silver or colloidal silver.

Preferably, the sanitising liquid is in liquid form in the cartridge.

Preferably, the method further comprises the step of measuring humidity of a space to be sanitised. Preferably, the method further comprises the step of activating an air purifier assembly configured to dehumidify air, in response to detecting that the humidity of the space is greater than a threshold value. Preferably, the method further comprises the step of continuously measuring the humidity of the space while the air purifier assembly is active. Preferably, the method further comprises the step of deactivating the air purifier assembly in response to the humidity of the space being measured to be below the threshold value. Preferably, the fan and air purifier assembly are controlled by a system controller. Preferably, the threshold value is between 70% and 90% humidity. Preferably, the threshold value is between 75% and 85% humidity. More preferably, the threshold value is 80% humidity.

Preferably, the air purifier assembly comprises an air filter and a dehumidifier.

Preferably, the duct is connected to the air purifier assembly.

Preferably, the housing comprises one or more openings formed therein. Preferably, the openings are formed about the air purifier assembly to allow the air purifier assembly to draw in air.

Preferably, the chemical vaporising assembly further comprises a fluid level indicator. Preferably, the fluid level indicator is a digital fluid level indicator. Preferably, the fluid level indicator digitally and visually indicates an amount of sanitising chemical composition in the cartridge. Preferably, the digital fluid level indicator electronically communicates the amount of sanitising chemical composition in the cartridge to the system controller. Preferably, the digital fluid level indicator electronically communicates the amount of sanitising chemical composition in the cartridge to the system controller in the form of an electric signal.

Preferably, the vapor is transported from the housing by a duct connecting the housing and the dispersion member.

Preferably, the system controller is further programmed to activate the air purifier assembly to dehumidify the air of a space to be sanitised.

Preferably, the sanitisation system further comprising a sensing assembly comprising at least one of: a humidity sensor, a temperature sensor and a sensor configured to detect an amount of vaporised sanitising chemical composition in the air

Preferably, the duct comprises a plurality of passageways, wherein a first passageway is configured to transport vapor from the vaporiser to the dispersion member and a second passageway is configured to transport air from the space to the air purifier assembly after a sanitisation cycle.

In some embodiments, the sanitisation system is installed in a lift, train, tram or ferry.

In another aspect, the invention provides a sanitisation system comprising a chemical vaporising apparatus for storing and vaporising a sanitising chemical composition, the chemical vaporising assembly comprising:
a housing configured to receive and retaining one or more sanitising chemical composition containing cartridges;
an air purifier assembly located within the housing, the air purifier assembly configured to purify the air and maintain a low humidity environment; and
a vaporiser located within the housing, the vaporiser configured to convert a sanitising chemical composition in the chemical containing cartridges into a vapor.

Preferably, the sanitisation system further comprises a power supply. Preferably, the power supply comprises a 12V power supply or 240V power supply. Preferably, the power supply is connected to one or more of the system controller, the fan, the vaporiser and the air purifier assembly.

Further features and advantages of the present invention will become apparent from the following detailed description.

### BRIEF DESCRIPTION OF THE DRAWINGS

By way of example only, preferred embodiments of the invention will be described more fully hereinafter with reference to the accompanying figures, wherein:

FIG. 1 illustrates a sanitisation system in accordance with an embodiment of the present invention;

FIG. 2 illustrates a perspective view of the chemical vaporising assembly of the sanitisation assembly of FIG. 1;

FIG. 3 illustrates an end view of the chemical vaporising assembly;

FIG. 4 illustrates a side view of the chemical vaporising assembly;

FIG. 5 illustrates a top view of the chemical vaporising assembly;

FIG. 6 illustrates the outlet assembly of the sanitisation system of FIG. 1;

FIG. 7 illustrates a cross-section of a duct of the outlet assembly;

FIG. 8 illustrates the dispersion member and fan of the outlet assembly;

FIG. 9 illustrates the fan of the outlet assembly;

FIG. 10 illustrates a block diagram of the system controller of the sanitisation system;

FIG. 10a illustrates another block diagram of the system controller of the sanitisation system;

FIG. 11 illustrates embodiments of the sanitisation system installed on a train;

FIG. 12 illustrates embodiments of the sanitisation system installed on a ferry;

FIG. 13 illustrates embodiments of the sanitisation system installed on a tram;

FIG. 14 illustrates embodiments of the sanitisation system installed on a lift;

FIG. 15 illustrates a front view of the chemical vaporising assembly of a sanitisation assembly according to an embodiment of the present invention;

FIG. 16 illustrates a side view of the chemical vaporising assembly of the sanitisation assembly of FIG. 15;

FIG. 17 illustrates the dispersion member of the sanitisation assembly of FIG. 15;

FIG. 18 illustrates a chemical cartridge and reservoir of the chemical vaporising assembly of FIG. 15;

FIG. 19 illustrates a vaporiser of the chemical vaporising assembly of FIG. 15; and

FIG. 20 illustrates a coiled heat line within the heat exchanger of the vaporiser shown in FIG. 19.

### DETAILED DESCRIPTION OF THE PREFERRED EMBODIMENTS

Embodiments of the sanitisation system described herein are predicated on the ability to efficiently and quickly sanitise a space to reduce the chance of transmission of viral and bacterial matter. Embodiments of the sanitisation system include a wall mounted unit housing liquid hydrogen peroxide (H₂O₂), and the mechanical and electrical components. The wall mounted unit is ideally only mountable in one orientation (i.e. upright). Ground, roof or ceiling mount may be supported by installers onsite using a frame or other support.

A duct extends vertically from the top of the wall mounted unit to carry the vaporised hydrogen peroxide to either one, or a series of, outlet assemblies located within the space. The outlet assemblies disperse the vapor effectively enough to cover up to a 300 sqm room though a daisy chain of outlet assemblies.

The outlet assembly and ducting may include an inlet and corresponding duct for return air to the wall mounted unit.

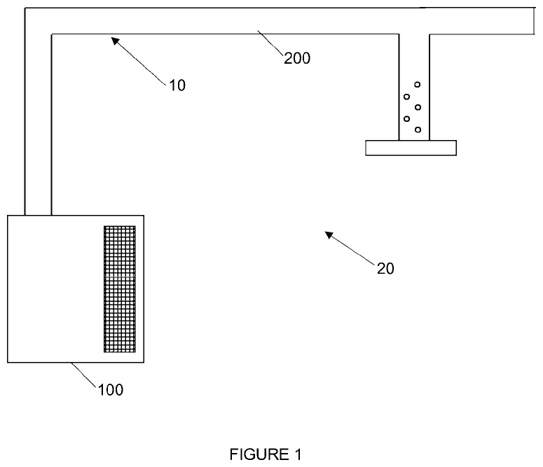

FIG. 1 illustrates a sanitisation system 10 adapted for substance dispersion according to a preferred embodiment of the present invention for sanitisation of a space (such as a bus, train or lift, for example).

The sanitisation system 10 includes a chemical vaporising assembly 100 connected to an outlet assembly 200 for dispersing a fog comprising a mixture of air and a vaporised sanitising chemical composition.

The chemical vaporising assembly 100 is configured to receive and store quantities of a sanitising chemical composition (preferably in a sealed container or receptable) and vaporise said chemical for dispersion as a fog into the space to be sanitised.

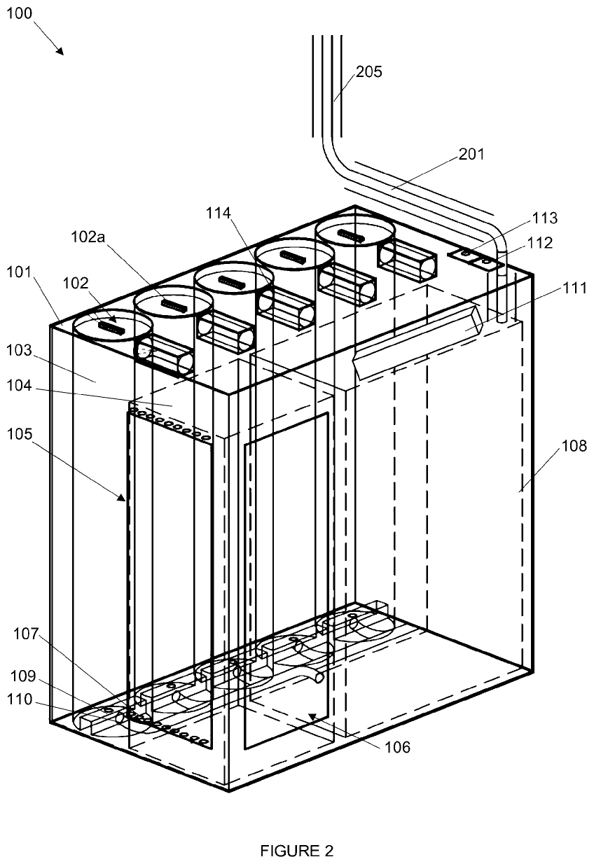

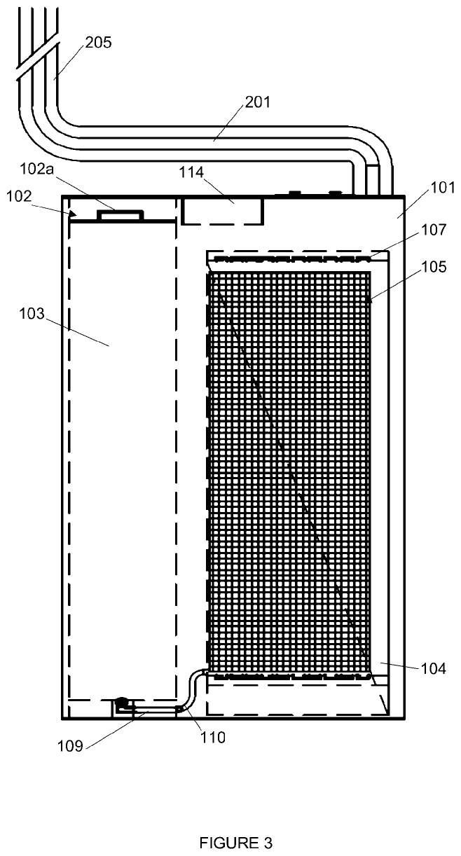

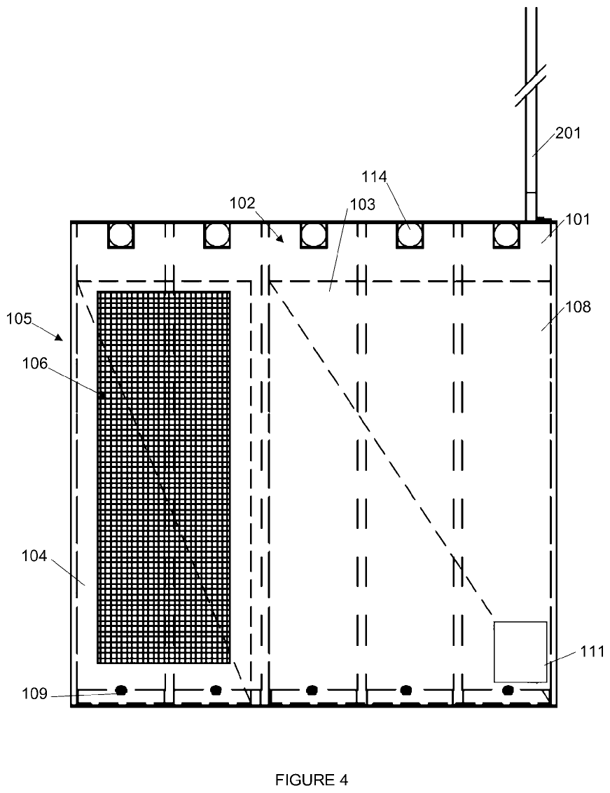

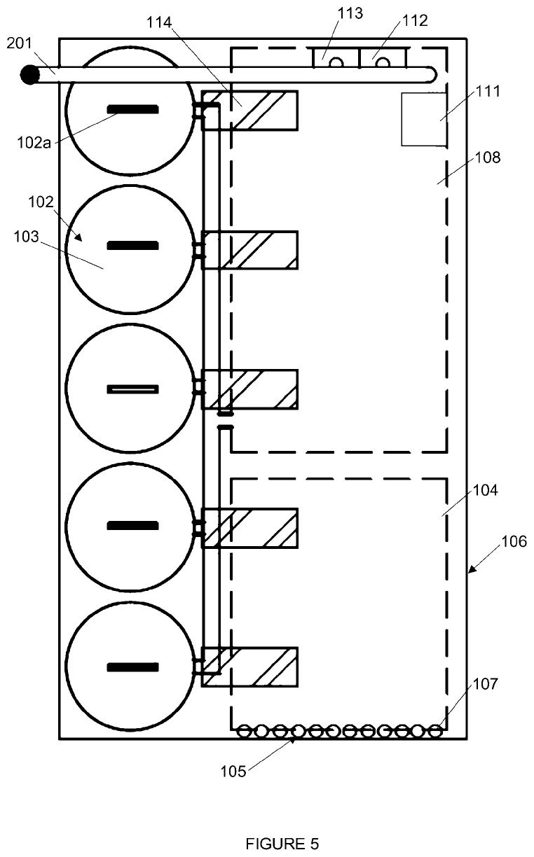

With reference to FIGS. 2-5, the chemical vaporising assembly 100 can be seen in greater detail.

The chemical vaporising assembly 100 comprises a housing 101 having five (5) cartridge receptacles 102, each having a chemical containing cartridge 103 located therein. The chemical containing cartridges 103 of the illustrated embodiment contain 35% mL/mL hydrogen peroxide (H₂O₂) which is known to be effective for sanitisation purposes. However, it will be appreciated that other chemicals and substances can be utilised, such as ionic silver, for example.

Furthermore, although the sanitising chemical composition is described as 35% mL/mL hydrogen peroxide, it should be recognised that embodiments of the invention can be used with up to 35% mL/mL hydrogen peroxide. In one embodiment, the sanitising chemical composition comprises between 0.1% mL/mL and 35% mL/mL hydrogen peroxide. In another particular embodiment, the Inventors have found that a concentration of no greater or no more than 10% mL/mL hydrogen peroxide is preferred as it reduces the risk of dermal irritation. The dispersion of vaporised hydrogen peroxide also presents a biological hazard through inhalation. To ameliorate the risk of inhalation of hydrogen peroxide, the sanitisation system 10 utilises the air purifier assembly 104 (fitted with scrubbing fans) to remove the hydrogen peroxide from the air before the sanitised space is to be used again. This will be explained in greater detail below.

The chemical vaporising assembly 100 also includes an air purifier assembly 104 located within the housing 101 which is in fluid communication with the atmosphere through two openings 105, 106 formed in the surface of the housing 101. The two openings 105, 106 are covered by gauze to assist in filtering the air and removing hydrogen peroxide. The air purifier assembly 104 includes a fan (preferably a high volume fan capable processing more than 1000 m³/h of air) which directs air through multiple filtering apparatus, including an ionizer, a high efficiency pre-filter 3-in-1 nano silver, charcoal/carbon mechanical filter and a line of sterilizing UVC lamps. The Inventors envision that filter blockages will need to be managed effectively to ensure the system can operate. Filter blockages can be detected through pressure differentials (difference between filter inlet and outlet), a venting flap (mechanical flap with switch which is enabled when airflow pressure is sufficient to provide an alert for blockages) and a mass air flow (MAF) sensor which measures reduced airflow using a hot wire MAF element.

The Inventors envision that the air purifier assembly 104 could include an electrically controlled actuator to attach the duct lines to the fan to allow efficient filtering of the air in the space (including hydrogen peroxide removal), regardless of the position of the housing 101 and thus exhaust any remaining hydrogen peroxide from the ducts. This would allow the housing 101 to be place outside or between multiple rooms or venues. In such an embodiment, the air purifier assembly 104 could exhaust into the space (i.e. recirculate) the prevent negative pressure from drawing contaminants back into the space from outside for installations requiring a high level of disinfection. Alternatively, the system may be ducted outside in order to draw in outside air to allow for a faster scrubbing cycle.

In some embodiments, the air purifier assembly 104 includes a catalytic agent (such as platinum or manganese oxide) to ensure all remaining hydrogen peroxide is converted to water and oxygen gas prior to contact with the mechanical filter.

In some further embodiments, the air purifier assembly includes a plurality of smaller fans, rather than a single fan, to minimise the footprint of the housing 101.

Located adjacent opening 105 is an ultraviolet (UV) light 107 which, when electrically powered, provides additional purification for the gases received into the air purifier assembly 104.

The housing 101 also includes a vaporiser 108 configured to convert the chemical contained in the cartridges 103 to be received therein into a vapor, gas or fine droplets. In operation, the vaporiser 108 is capable of converting the liquid H₂O₂ into vaporised hydrogen peroxide (commonly referred to as VHP) having aerosol droplets with a diameter of between 5 and 15 microns. In a preferred embodiment, the vaporiser 108 includes a fogger or induction heater which vaporises the sanitising chemical composition for dispersion into a space. Specifically, the vaporiser 108 may take the form of a metallic heat exchanger. In operation, the vaporiser 108 heats the hydrogen peroxide to at least 150° C. (and up to 220° C.) in a closed-loop environment to ensure that no liquid hydrogen peroxide is dispersed.

At the base of each receptacle 102 is a valve 109 connected to a passage 110 to connect the receptable 102 to the vaporiser 108 and thereby allow the chemical in the cartridges 103 to be transferred from the cartridge 103 to the vaporiser 108 by a gravity feed.

At a top of each receptacle 102 is a locking mechanism 102a. The locking mechanism 102a may take the form of a lock and key or an electronic safe-type mechanism accessible by a PIN. This ensures the chemical cartridges can only be accessed and removed from the housing 101 by those authorised to do so.

Furthermore, the chemical vaporising assembly 100 includes a computing device in the form of a system controller 111 for controlling the chemical vaporising assembly 100 and in particular the vaporiser 108.

FIG. 10 shows a block diagram of the electronic and mechanical components of the system 10.

The system controller 111, shown in FIG. 10a, includes a processor 111a in electrical communication with digital memory 111b and a wireless transceiver 111c (taking the form of a Bluetooth or Wi-Fi transceiver, in some embodiments) for communicating with an external monitoring system 300.

The system controller 111 also includes a USB port 111d in electrical communication with the CPU 111a for communication with external devices that are plugged into the USB port 111d (for example, USB storage drives).

The chemical vaporising assembly 100 additionally includes a power supply in the form of a 12V power supply 112 and a sensing assembly 113 for detecting environmental conditions including the humidity of the space to be sanitised. In some embodiments, the power supply comprises a 240V power supply which suitable for embodiments to be installed in lifts and standard building enclosures.

Conveniently, the housing 101 also includes a digital fluid level indicator 114 for digitally monitoring and visually indicating an amount of chemical remaining in each of the chemical cartridges 103. Alternatively, in some embodiments, a non-magnetic float level can be used to monitor cartridge fluid level.

Preferably, the housing 101 has a slimline form factor to minimise disruption to the space it is installed in and has an ingress protection rating of 54 or higher.

As can be seen in FIGS. 1 and 2, the outlet assembly 200 is connected to the chemical vaporising assembly 100. The outlet assembly 200 includes an enclosed outlet duct 201 in the form of a 50 mm diameter pipe which is fluidly connected to the vaporiser 108 to transport the vaporised chemical from the chemical vaporising assembly 100 to the space to be sanitised. It should be noted that the diameter of the pipe of enclosed outlet duct 201 may be between 20 mm and 55 mm.

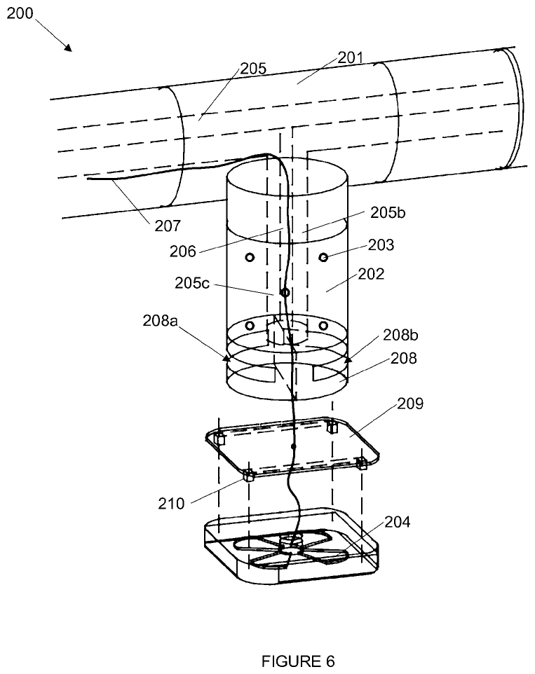

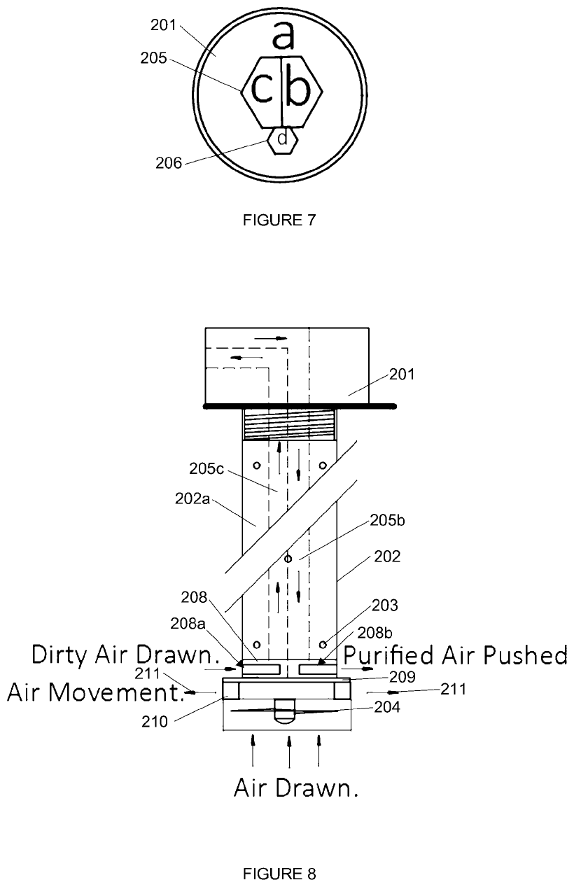

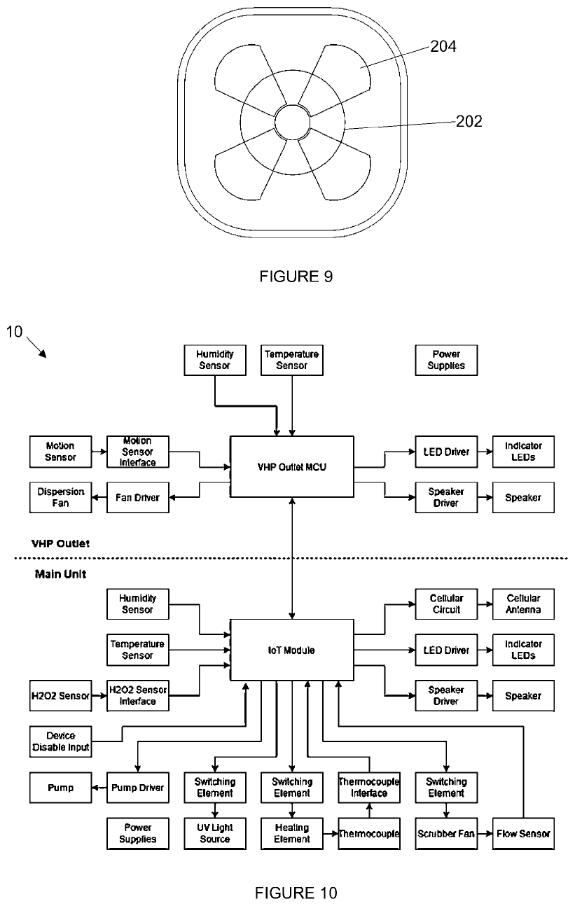

As can be seen in the figures, the enclosed outlet duct 201 includes a second duct 205 in the form of a 25 mm conduit therein and a third duct 206 adjacent second duct 205. A cross-section of the outlet duct 201, second duct 205 and third duct 206 are shown in FIG. 7. It can be seen from the cross section that outlet duct 201 surrounds hexagonal second duct 205 and hexagonal third duct 206. The second duct is divided into two duct portions, 205b and 205bc. Duct Portion 205b carries air from the air purifier assembly 104 to the space 20, and duct portion 205c carries air from the space 20 to the air purifier assembly 104. Third duct 206 encloses a wire 207 therein for supplying electrical power to the fan 204, which will be described in more detail below (this can be seen in FIG. 6).

The enclosed outlet duct 201 of the outlet assembly 200 connects to a dispersion member 202. Preferably, the dispersion member 202 is an elongate dispersion member.

In the illustrated embodiment, the outlet duct 201 and dispersion member 202 are threadedly connected, as shown in FIG. 8, such that the outlet duct 201 and dispersion member 202 are screwed together. However, it is envisioned that the outlet duct 201 and dispersion member 202 can be connected in a number of known ways. While the outlet assembly 200 is shown and described as having only one dispersion member 202, it should be recognised that the outlet assembly 200 may have a plurality of dispersion members connected thereto.

The dispersion member 202 comprises a plurality of apertures 203 formed therein to allow the vapor transported from the chemical vaporising assembly 100 to be dispersed into the space to be sanitised. The dispersion member 202 is preferably made from copper as copper is known to be non-reactive.

In some embodiments, the apertures 203 formed in the dispersion member 202 are 2 mm in diameter and are placed about the circumference of the cylindrical dispersion member 202 to provide even dispersion of the fog.

Connected to the end of the dispersion member 202 is a fan 204 which can operate in both an intake and output capacity to assist with vapor dispersion into the space 20. The fan 204 can be made from metal but is preferably made from plastic. The fan 204 includes a backing plate 209 having spacers 210, located between the dispersion member 202 and the fan 204.

Located at an opposing end of the dispersion member 202 between the body of the dispersion member 202 and the fan 204 is a slotted head 208.

The slotted head 208 is threadedly connected to the dispersion member 202 and includes two openings 208a, 208b.

First opening 208a, which is connected to and is in fluid communication with duct portion 205c, draws air (dirty or unpurified air having hydrogen peroxide therein) into duct portion 205c of second duct 205 from the space 20 to be transported along duct portion 205c back to the purifier 104.

Second opening 208b, which is connected to and is in fluid communication with duct portion 205b, receives purified air transported from the purifier 104 through duct portion 205b and expels and disperses that purified air into space 20.

An example of operation of the sanitisation system 10 will now be provided.

The sensing assembly 113 including a humidity sensor 113a and a temperature sensor 113b, under the instruction of the processor 111a of the system controller 111, detects the humidity and temperature of the space 20 to be sanitised and outputs an electrical signal that is converted by the Analog to Digital Converter 113z and read by the processor as a value indicative of the humidity. In high humidity environments, the efficacy of the system is reduced. Thus, if a high humidity (approximately 80% humidity or higher) is detected, the air purifier assembly 104, also under the instruction of the processor 111a, is activated and run until the humidity sensor 113a indicates that the humidity of the air has reached an optimal or acceptable level below a threshold value. In some embodiments, the threshold value is between 70% and 90% humidity. In some further embodiments, the threshold value is between 75% and 85% humidity. In a more preferable embodiment, the threshold value is 80% humidity.

The system 10 also includes a hydrogen peroxide sensor 113c to monitor the concentration of VHP in the air and a motion sensor 113d to detect people within the space to be sanitised to ensure that the hydrogen peroxide is not dispersed within the space until the space has been entirely vacated.

Additionally, the system 10 can include an interface connecting sensors 113e, or a chain of sensors to detect open windows, doors or threshold detection (like shop welcome chimes) will be included. This allows an interlock to prevent or stop treatment from occurring when spaces are uncontained, e.g. open to outside environment or public places or where presence of a person is detected when not expected. Furthermore, this allows the system 10 to cease treatment and rapidly vent/scrub to reduce danger, particularly in areas where motion sensors are not feasible, such as with view of outside the space where there may be movement.

In addition to the above, the system 10 includes a vapor pressure sensor 113f (or pressure switch in addition to a pressure relief valve). The vapor pressure sensor 113f is configured to detect blockage in pressurised parts of the system 10 and, through communication with processor 111a, can prompt an emergency stop of vaporiser 108 and pump to prevent an explosion due to VHP thermal decompression.

The sensing assembly 113 further includes an IOT (Internet of Things) assembly 114.

Subsequently, upon the humidity of the space 20 being detected to be conducive to operation of the system 10, the processor 111a instructs the valve 109 to open and allow an amount of the chemical in the chemical cartridges 103 to flow to the vaporiser 108 which transforms the chemical into a vapor.

As the chemical is vaporised, the vapor travels out of the chemical vaporising assembly 100 via the enclosed outlet duct 201 of the outlet assembly 200.

Travelling along the enclosed outlet duct 201, the vapor eventually reaches the internal cavity 202a of the dispersion member 202.

The fan 204, under instruction of the processor 111a, begins operating in an air intake mode to draw in air from the space 20 toward the blades of the fan 204. As indicated by arrow 211, the air that is drawn in by the fan 204, impacts the backing plate 209 and is thus pushed outwardly and sideways away from the fan . This causes the vapor dispersed out through the apertures 203 of the dispersion member 202 and air pushed away from the fan 204 to mix and diffuse into the space 20. Thus commencing the sanitisation process.

The dispersion of the mixture of air and vapor into the space 20 is envisioned by the Inventors to run for approximately 2 to 4 minutes depending on the size of the space 20 to be sanitised. Of course, it should be appreciated that the total run time can be any length of time.

To allow air quality to return to a level that is safe for occupation, the Inventors envision that approximately 10 minutes should be allowed from the cessation of the sanitisation until a person enters the space 20. This is envisioned to allow sufficient time for the vaporised chemical to be dispersed, contact the surfaces of the space 20 and be vented so that the space 20 can be safely used again. Again, this time may vary based on the environmental factors and dimensions of the space.

The processor 111a then activates the air purifier assembly 104 to further purify the air and remove remaining germs, bacteria, viruses, diseases and the like. The purification of the air can be further aided by the activation of the UV light 107. UV light is known to have sterilising properties.

In some embodiments, the air purifier assembly 104 may remain active to maintain environmental conditions (i.e. humidity) at a level conducive to optimal operation of the system 10.

Finally, the processor 111a, in communication with the fluid level indicators 114, records the amount of chemical utilised from each cartridge and records this data in memory 111b for auditing and reporting purposes. The recorded data may be accessed and downloaded via the USB port 111d in electrical communication with the CPU 111a.

The system 10 also stores data relating to temperature, humidity, sanitisation cycle status, service logs and filter status collected from various sensors described herein.

The data can also be wirelessly communicated via the wireless transceiver 111c to an external monitoring system 300, such as a central operations system for public transport or an office building.

Following the completion of the above process, the air and surfaces can reliably be considered to be substantially free from germs, bacteria, viruses and diseases.

Embodiments of the invention as described herein can be used to sanitise frequently used areas of buildings (such as common kitchen areas and elevators, for example) and public transport vehicles (such as buses and trains, for example). Embodiments of the invention can also be used in outdoor environments.

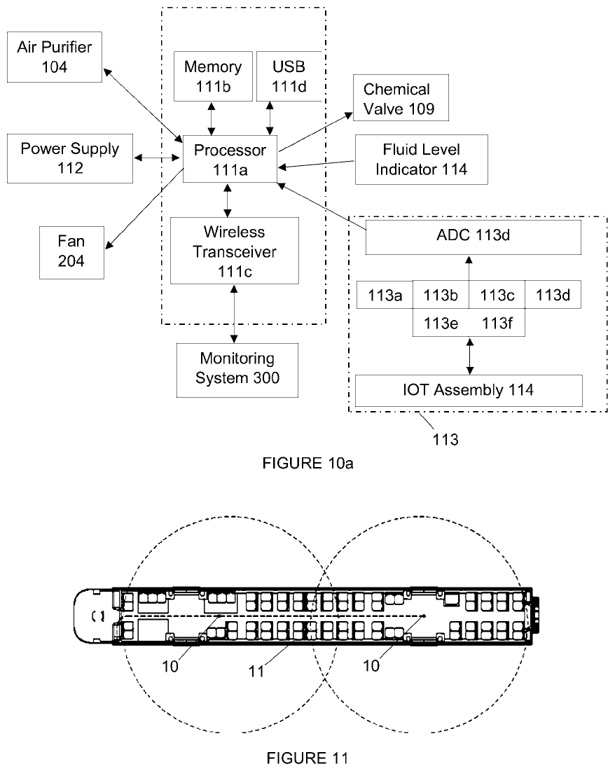

Some exemplary uses of embodiments of the invention described herein are shown in FIGS. 11-14.

FIG. 11 illustrates two sanitisation systems 10 installed on a train 11.

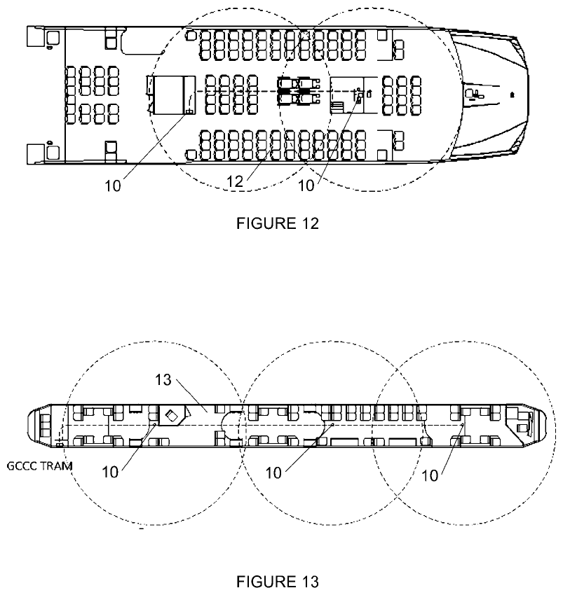

In another example, FIG. 12 illustrates two sanitisation systems 10 installed on a ferry 12.

In a further example, FIG. 13 illustrates three sanitisation systems 10 installed on a tram 13.

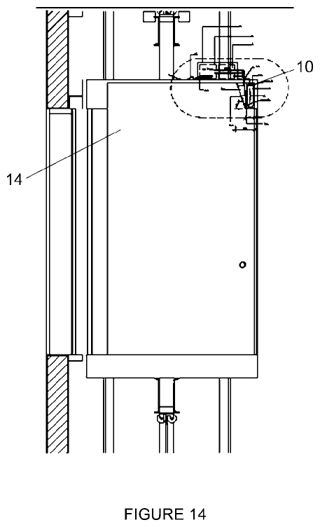

In another example, FIG. 14 illustrates a sanitisation system 10 installed in a lift 14.

In use, the system 10 performs a sanitisation cycle when safe to do so. The system 10 can be programmed to monitor a number of variables to ensure that the sanitisation cycle is only initiated when safe to do so, but to also immediately cease the sanitisation cycle if one or more safety parameters are no longer satisfied.

An example of the above, includes the system 10 determining that a plurality of safety interlocks are satisfied. The safety interlocks can include: (i) humidity of the space is below an acceptable level (discussed above; (ii) all doors and windows are closed (determined by sensors); (iii) no motion detected in the space (detected by motion sensors); and (iv) sufficient hydrogen peroxide in reservoir/cartridges.

Once the safety interlocks are satisfied, the system 10 creates a sanitising dry fog by pumping liquid 10% hydrogen peroxide via a peristaltic pump (a heating line) into the vaporiser 108 (having a metallic heat exchanger) and heating it to at least 150° C. to convert the liquid chemical mixture to dry fog particles of less than 15 microns. This process is known as “hot fogging”. The pressurized fog is then transferred through ducted channels and released via at least one (but potentially multiple) outlet assembly 200.

The fan 204 of the outlet assembly 200 assists with circulating the VHP around the space evenly and thoroughly to sanitise the space effectively.

Once the sanitisation cycle is completed, the air purifier assembly 104 is activated to purify/scrub the air and remove the harmful hydrogen peroxide from the space prior to people entering the space.

Preferably, the system 10 runs the air purifier assembly 104 for between 5 and 10 minutes and/or until the amount of hydrogen peroxide in the air in the space returns to an acceptable level such that being in the space is unlikely to cause harm to any people in the space.

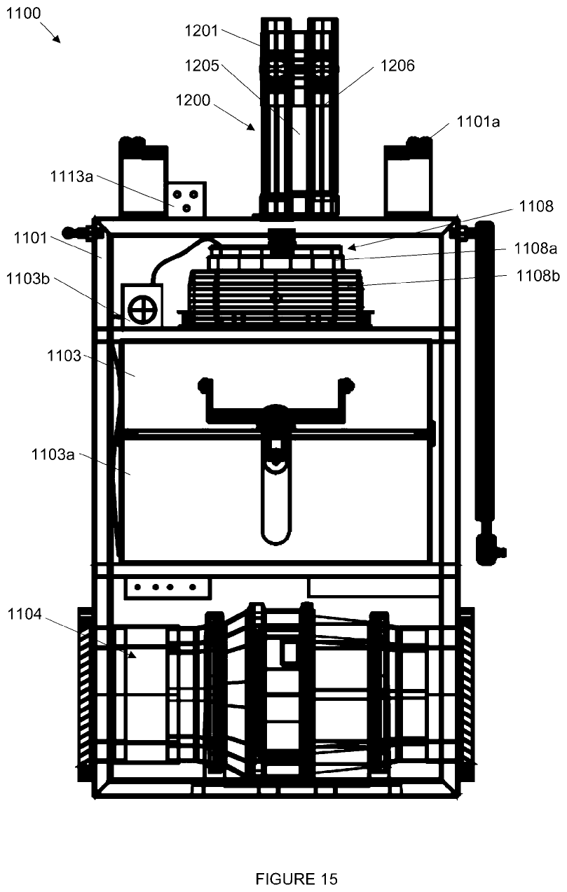

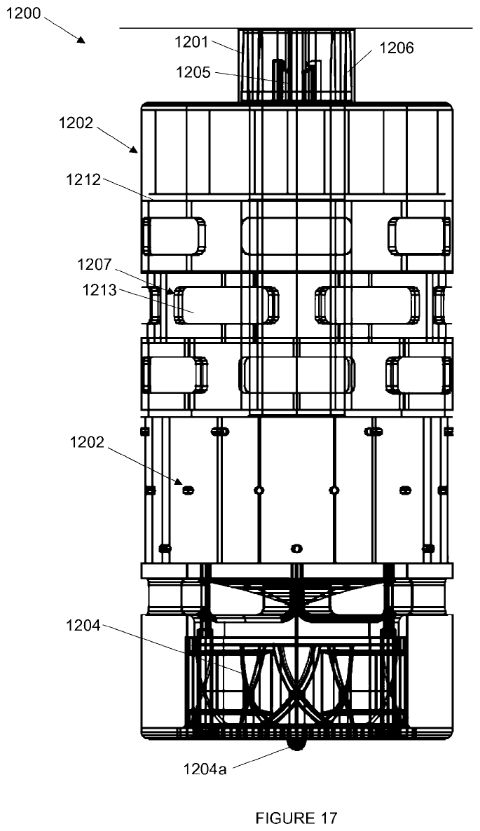

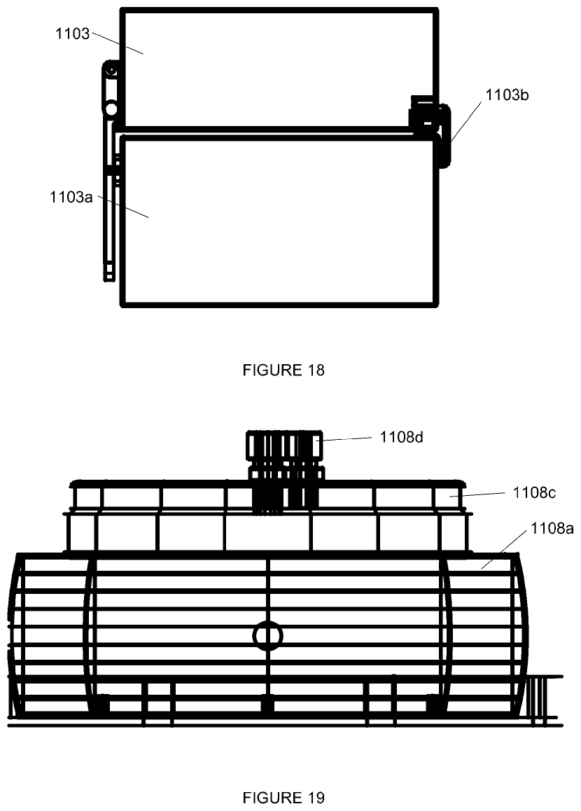

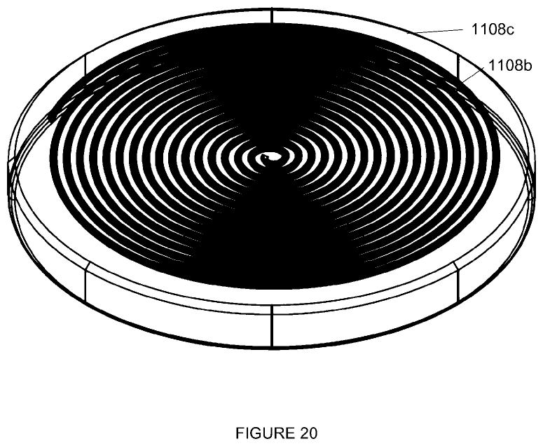

In an alternative embodiment shown in FIGS. 15-20, chemical vaporising assembly 1100 includes a cartridge 1103 sits atop a reservoir 1103a in housing 1101. The contents (i.e. the hydrogen peroxide) of the cartridge 1103 drain into the reservoir 1103a through valve 1103b via gravity and the contents are then pumped via a pump 1103b (preferably via a peristaltic pump) from the reservoir 103a to the vaporiser 1108 having an induction plate 1180a and coiled heat line 1108b within heat exchanger 1108c. The coiled heat line 1108b is connected to a release nozzle 1108d which is, in turn, connected to the outlet assembly 1200. It should be understood that cartridge 1103, housing 1101 and vaporiser 1108 are substantially similar to cartridge 103, housing 101 and vaporiser 108 described above.

The coiled heat line 1108b assists with building pressure and propulsion of the VHP from the vaporiser 1108 through outlet assembly 1200.

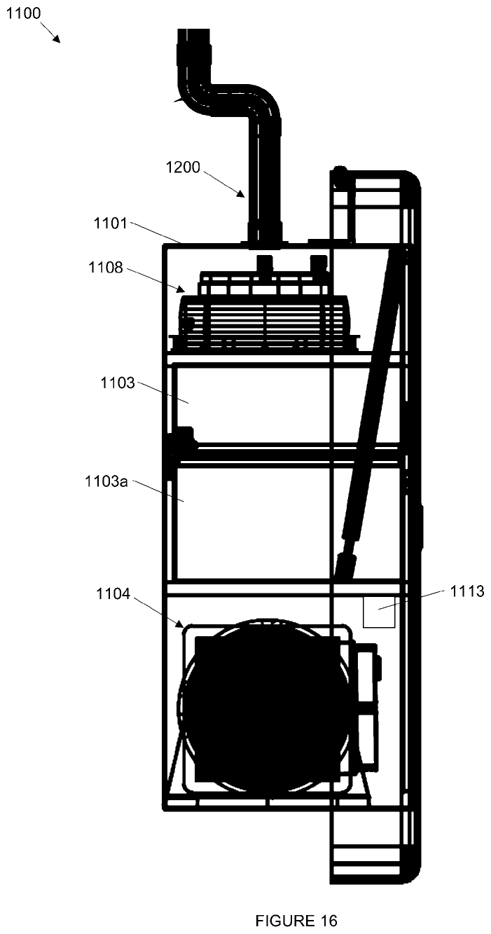

Referring to FIGS. 15 and 16, there is shown a chemical vaporising assembly 1100 having a housing 1101 which houses the cartridge 1103 atop the reservoir 1103a which is connected to the vaporiser 1108. The vaporiser 1108 is connected to the outlet assembly 1200. The outlet assembly 1200 includes a first duct 1201, second duct 1205 and third duct 1206.

First duct 1201 is a VHP carrying duct which is connected to release nozzle 1108d and transports VHP from the vaporiser 1108 to the dispersion member 1202 for dispersion within the space through apertures 1203 formed in the cylindrical body 1212 of the dispersion member 1202.

Second duct 1205 is an air return duct in communication with inlets 1213 formed in the cylindrical body 1212, which can also be fitted with UV lights 1207 for additional air purification, which carries air from the space to the housing 1101 and the air purifier assembly 1104 for filtering and purification.

Third duct 1206 is a power and data conduit for providing power to the fan 1204 located in the cylindrical body 1212 of the dispersion member 1202 and for receiving data from the sensors 1204a located in the dispersion member 1202. The sensors 1204a can include one or more of a humidity sensor, a hydrogen peroxide sensor and one or more cameras.

In some embodiments, a gauze is applied over the fan 1204 to provide an additional filter.

Located at the bottom of the housing 1101 is an air purifier assembly 1104 (substantially similar to air purifier assembly 104 described above) which includes one or more of an ionizer, a fan, air filters and UV lights.

Also located in the housing 1101 is a sensing assembly 1113. The sensing assembly 1113, similar to sensing assembly 113, includes a humidity sensor, a temperature sensor, a motion sensor, a VHP or hydrogen peroxide sensor (sensing VHP parts per million) along with a CPU and processing assembly.

Sitting atop the housing 1101 are an electronic lock 1101a for securing the housing 1101 and its contents, and a camera array 1113a configured to detect one or more of body temperature of any people in the space, the number of people in the space and the distance between said people for monitoring adherence with social distancing requirements that may be in place.

The heating line is to be a thermally conductive material with a sufficient length to ensure the chemical is entirely vaporised before entering the ducting. The Inventors have found that coiling the heating within the heat exchanger reduces size.

In some embodiments, the heat exchanger of the vaporiser takes the form of a domestic grade induction heater to heat and control the temperature of the heat exchanger. The heat exchanger can also include over temperature protection or pressure relief in the heat exchanger or vapor chambers. In an embodiment, a thermo block type design be employed to simplify the thermal temperature and power control. Importantly, all parts of the system which are in contact with H₂O₂ should be manufactured from materials which are compatible with this chemical in liquid state and will not degrade in this environment. Suitable materials include but may not be limited to high purity aluminium, low carbon stainless steel, ABS, HDPE, LDPE, polycarbonate and PVC.

The system 10 operates on the assumption that 500 mL of hydrogen peroxide will be required to be vaporised and released into the space over a maximum period of 25 minutes. It is envisioned that the amount of hydrogen peroxide could be reduced to 200 mL over a period of 5-10 minutes in some embodiments.

Vaporising 200 mL of 10% H₂O₂ in 10 minutes will require approximately 1500 W of thermal heat to be applied for that time.

The VHP of the system is cooled quickly at ambient pressure before entering the environment to reduce the likelihood of condensation. When cooled under pressure, such as if a long pressurised VHP tube was to extend several meters from the main unit to the outlet assembly, then condensation of the vapour may occur.

Alternative methods to thermal fogging may include cold fogging processes such as ULV. Thermal fogging reliably produces smaller particle sizes (“dry fog” Less the 15 Microns) as opposed to cold foggers like ULV, which can produce wet fog. Wet H₂O₂ fog could cause bleaching to some materials or be otherwise harmful. Thermal fogging carries other risks associated with heat and can cause explosions with high concentration aqueous hydrogen peroxide solutions.

In some embodiments, humidity control may be utilised to ensure the system operates only in suitable environments. The presence and decomposition of VHP in air will increase the relative humidity. For example, at 10° C., 200 mL of water will increase the relative humidity of a 100m3 room from 60% to about 80% 4. As many installations will include air-conditioning, it is envisioned that a dehumidifier will not be necessary in all embodiments of the system, but may be included where necessary. However, a humidity sensor is preferably included to prevent or stop the treatment of a space in the event the humidity rises above set levels.

In one embodiment, a distilled water flush may be performed as part of regular maintenance (proposed to be 4-6 monthly). In an embodiment, hydrogen peroxide would be drained from the reservoir and a regular treatment cycle would run with distilled water rather than chemical in order to flush the system.

Advantageously, embodiments of the present invention provide consistent sanitisation of areas and spaces to lower the risk of transmission of infections diseases, bacteria and viruses.

In a further advantage, embodiments of the system described herein are envisioned to be effective at sanitising spaces up to 300 sqm in size. However, the Inventors envision that some embodiments could treat larger space by operating the system for longer.

The systems illustrated and described herein utilise a physical device, which on command, delivers a dry vaporised fog of hydrogen peroxide in order to sanitise a space and thereby reduce the presence, and in turn, the transmission of bacteria, viruses, spores and other disease.

The device is intended to be installed in a space, and blends in with the surroundings, in order to appear part of the day to day routine of the space and thereby raise public confidence in cleanliness of the space.

In this specification, adjectives such as first and second, left and right, top and bottom, and the like may be used solely to distinguish one element or action from another element or action without necessarily requiring or implying any actual such relationship or order. Where the context permits, reference to an integer or a component or step (or the like) is not to be interpreted as being limited to only one of that integer, component, or step, but rather could be one or more of that integer, component, or step, etc.

The above detailed description of various embodiments of the present invention is provided for purposes of description to one of ordinary skill in the related art. It is not intended to be exhaustive or to limit the invention to a single disclosed embodiment. As mentioned above, numerous alternatives and variations to the present invention will be apparent to those skilled in the art of the above teaching. Accordingly, while some alternative embodiments have been discussed specifically, other embodiments will be apparent or relatively easily developed by those of ordinary skill in the art. The invention is intended to embrace all alternatives, modifications, and variations of the present invention that have been discussed herein, and other embodiments that fall within the spirit and scope of the above described invention.

In this specification, the terms ‘comprises’, ‘comprising’, ‘includes’, ‘including’, or similar terms are intended to mean a non-exclusive inclusion, such that a method, system or apparatus that comprises a list of elements does not include those elements solely, but may well include other elements not listed.

Throughout the specification and claims (if present), unless the context requires otherwise, the term “substantially” or “about” will be understood to not be limited to the specific value or range qualified by the terms.

What is claimed is:

1. A sanitisation system, the sanitisation system comprising:
a chemical vaporising apparatus for storing and vaporising a sanitising chemical composition, the chemical vaporising assembly comprising:
a housing configured to receive and retaining one or more cartridges containing a sanitising chemical composition;
a vaporiser located within the housing, the vaporiser configured to receive and convert a sanitising chemical composition in the chemical containing cartridges into a vapor;
an air purifier assembly located within the housing, the air purifier assembly configured to purify the air by removing vaporised sanitising chemical composition and maintain a low humidity environment; and
an outlet assembly comprising:
a duct connected to the chemical vaporising assembly for transporting the vapor therein;
an elongate dispersion member having a first end connected to the duct, the elongate dispersion member having a plurality of apertures formed therein for dispersing vapor therethrough; and
a fan located at a second end of the elongate dispersion member distal to the first end of the elongate dispersion member for drawing air into the dispersion member; and
a system controller programmed to activate the fan to draw air into the elongate dispersion member for mixing with the vapor and dispersal through the plurality of apertures formed in the elongate dispersion member.

2. The sanitisation system according to claim 1, wherein the sanitising chemical composition comprises liquid hydrogen peroxide.

3. The sanitisation system according to claim 2, wherein the sanitising chemical composition comprises up to 35% mL/mL hydrogen peroxide or between 0.1% mL/mL and 35% mL/mL hydrogen peroxide or no more than 10% mL/mL hydrogen peroxide.

4. The sanitisation system according to claim 1, wherein the fan and air purifier assembly are controlled by the system controller.

5. The sanitisation system according to claim 1, wherein the air purifier assembly comprises an air filter and/or a dehumidifier and/or UV lights and/or a fan for drawing air.

6. The sanitisation system according to claim 1, wherein the duct is connected to the air purifier assembly.

7. The sanitisation system according to claim 1, wherein the housing comprises one or more openings formed therein and the one or more openings are formed about the air purifier assembly to allow the air purifier assembly to draw in air.

8. The sanitisation system according to claim 1, wherein the chemical vaporising assembly further comprises a fluid level indicator.

9. The sanitisation system according to claim 8, wherein the fluid level indicator digitally and/or visually indicates an amount of sanitising chemical composition in the cartridge and electronically communicates the amount of sanitising chemical composition in the cartridge to the system controller.

10. The sanitisation system according to claim 1, wherein the vapor is transported from the housing by the duct connecting the housing and the dispersion member.

11. The sanitisation system according to claim 1, wherein the system controller is further programmed to activate the air purifier assembly to dehumidify and/or remove sanitising chemical from the air of a space to be sanitised.

12. The sanitisation system according to claim 1, the sanitisation system further comprising a sensing assembly comprising at least one of: a humidity sensor, a temperature sensor and a sensor configured to detect an amount of vaporised sanitising chemical composition in the air.

13. The sanitisation system according to claim 1, wherein the duct comprises a plurality of passageways, wherein a first passageway is configured to transport vapor from the vaporiser to the dispersion member and a second passageway is configured to transport air from the space to the air purifier assembly after a sanitisation cycle.

14. A sanitisation system comprising a chemical vaporising apparatus for storing and vaporising a sanitising chemical composition, the chemical vaporising assembly comprising:
a housing configured to receive and retaining one or more sanitising chemical composition containing cartridges;
a vaporiser located within the housing, the vaporiser configured to receive and convert a sanitising chemical composition in the chemical containing cartridges into a vapor; and
an air purifier assembly located within the housing, the air purifier assembly configured to purify the air by removing vaporised sanitising chemical composition from the air and maintain a low humidity environment.

15. A method for sanitisation, the method comprising the steps of:
generating a vapor from a sanitising chemical composition by a vaporiser in a housing;
transporting the vapor to a dispersion member having a plurality of apertures formed therein; and
activating a fan to draw air into the dispersion member to mix the air and the vapor and disperse the mixture into a surrounding space through the apertures.

16. The method of claim 15, wherein the method further comprises the step of measuring humidity of a space to be sanitised.

17. The method of claim 16, wherein the method further comprises the step of activating an air purifier assembly configured to dehumidify air, in response to detecting that the humidity of the space is greater than a threshold value.

18. The method of claim 17, wherein the method further comprises the step of continuously measuring the humidity of the space while the air purifier assembly is active.

19. The method of claim 16, wherein the method further comprises the step of deactivating the air purifier assembly in response to the humidity of the space being measured to be below the threshold value.

20. The method of claim 17, wherein the threshold value is between 70% and 90% humidity, or the threshold value is between 75% and 85% humidity or the threshold value is 80% humidity.
